(19) United States
(12) Patent Application Publication
(10) Pub. No.: US 2008/0234213 A1
(43) Pub. Date: Sep. 25, 2008

(54) Oncogenic regulatory RNAs for diagnostics and therapeutics

(76) Inventors: Matthias Wabl, San Francisco, CA (US); Bruce Wang, Mountain View, CA (US)

Correspondence Address: PERKINS COIE LLP, P.O. BOX 1208, SEATTLE, WA, 98111-1208, US

(21) Appl. No.: 11/515,263

(22) Filed: Sep. 1, 2006

Related U.S. Application Data
(60) Provisional application No. 60/713,674, filed on Sep. 2, 2005.

(51) Int. Cl. A61K 31/70; C12Q 1/68; C07H 21/00; A61P 43/00

(52) U.S. Cl. 514/44; 435/6; 536/23.1

(57) ABSTRACT

A method of identifying regulatory RNAs, including miRNAs, using insertional mutagenesis to generate tumors in mice and determining the human orthologs is disclosed. Further, specific miRNA sequences are identified. The causal nature and expression patterns of these regulatory RNAs and miRNAs in human tumors demonstrate their utility in diagnosis and therapy of cancer. Furthermore, a set of co-mutations that act in conjunction with miRNAs in tumor formation is disclosed.

This application claims the benefit of U.S. Provisional Application Ser. No. 60/713,674, filed Sep. 2, 2005, which is incorporated herein by reference.

### TABLES 1A, 1B, 1C, 2A AND 1B

The present application incorporates by reference Tables 1A, 1 B, 2A, and 2B contained on one compact disc filed concurrently herewith, which compact disc is labeled “Copy 1-Tables 1A-2B”. The details of Tables 1A-2B are further described later in this disclosure. This compact disc was created on 2 Sep. 2005 and is 680 MB in size. The CD contains three files labeled Table 1A.doc (88 KB), Table 1B.doc (5721 KB), and Table 2A-2B.doc (223 KB). These files are expressly incorporated herein by reference.

### I. REFERENCES

The following references are cited below in support of the background of the invention or methods employed in practicing the invention.
1. McManus, Immunity, 21:747-756 (2004).
2. Bartel, Cell, 116:281-297 (2004).
3. Cai et al., Rna, 10:1957-1966 (2004).
4. Lee et al., Embo J, 23:4051-4060 (2004).
5. Lee et al., Nature, 425:415-419 (2003).
6. Bernstein et al., Nature, 409:363-366 (2001).
7. Calin et al., Proc Natl Acad Sci USA, 99:15524-15529 (2002).
8. Calin et al., Proc Natl Acad Sci USA, 101:2999-3004 (2004).
9. Calin et al., Proc Natl Acad Sci USA, 101:11755-11760 (2004).
10. Griffiths-Jones, Nucleic Acids Res, 32:D109-D111 (2004).
11. Bentwich et al., Nat Genet, 37:766-770 (2005).
12. Ota et al., Cancer Res, 64:3087-3095 (2004).
13. He et al., Nature, 435:828-833 (2005).
14. Akagi et al., Nucleic Acids Res, 32:D523-D527 (2004).
15. Collier et al., Nature, 436:272-276 (2005).
16. Dupuy et al., Nature, 436:221-226 (2005).
17. Suzuki et al., Nat Genet, 32:166-174 (2002).
18. Lund et al., Nat Genet, 32:160-165 (2002).
19. Hwang et al., Proc Natl Acad Sci USA, 99:11293-11298 (2002).
20. Mikkers et al., Nat Genet, 32:153-159 (2002).
21. Li et al., Nat Genet, 23:348-353 (1999).
22. Lovmand et al., J Virol, 72:5745-5756 (1998).
23. van Lohuizen et al., Cell, 65:737-752 (1991).
24. Nusse et al., Cell, 31:99-109 (1982).
25. Nusse et al., Nature, 307:131-136 (1984).
26. Berezikov et al., Cell, 120:21-24 (2005).
27. Tanzer et al., J Mol Biol, 339:327-335 (2004).
28. Lim et al., Science, 299:1540 (2003).
29. Xie et al., Nature, 434:338-345 (2005).
30. Weng et al., Science, 306:269-271 (2004).
31. Justice et al., Mamm Genome, 11:484-488 (2000).
32. Hallberg et al., J Virol, 65:4177-4181 (1991).
33. Nielsen et al., J Virol, 70:5893-5901 (1996).
34. Sørensen et al. J Virol, 70:4063-4070 (1996).
35. Kim et al., J Virol, 77:2056-2062 (2003).
36. Walther et al. Genome Res, 11:875-888 (2001).
37. Hofacker et al., Chemie, 125:167-148 (1994).
38. Zuker et al., Nucleic Acids Res, 9:133-148 (1981).
39. McCaskill, Biopolymers, 29:1105-1119 (1990).
40. Shtivelman et al., Proc Natl Acad Sci USA, 86: 3257-3260, (1989).
41. Arya et al., Expert Rev Mol Diagn: 5: 209-219 (2005).

### II. BACKGROUND

MicroRNAs (miRNAs) are small, non peptide-coding RNAs that regulate gene expression in a variety of physiological and developmental processes^{1,2}. In the biogenesis of miRNAs, primary miRNA transcripts (pri-miRNAs) are first generated by RNA polymerase II^{3,4} and are then further processed like messenger RNA transcripts with the addition of a 5′ cap structure and poly A tail. Because of this, the pri-miRNA transcripts can be found in standard cDNA libraries.

The primary transcript can be over 3 kb long and adopt one or several stem-loop structures which are subsequently processed by the enzymes Drosha⁵ and/or Dicer⁶ to generate mature miRNA. The mature miRNAs are generally 18 to 24 nucleotides long and are incorporated into the RNA-induced silencing complex (RISC), which inhibits translation by binding to similar, but not identical sequences, of the 3′ untranslated region of mRNA. If the interaction is perfectly complementary, the miRNA may act as small inhibitory RNA (siRNA) leading to the degradation of the target mRNA. Often, a pri-miRNA transcript is polycistronic, i.e., one pri-miRNA transcript yields several different miRNAs. Further, miRNAs can be found within primary gene transcripts.

Dysregulated miRNA expression has been postulated to contribute to lymphoma formation in humans⁷⁻⁹. The miRNA registry¹⁰ currently contains over 200 examples that are shared between humans and mice; another 89 miRNAs are found only in primates¹¹. Of these, one miRNA cluster has been demonstrated to be overexpressed in human B cell lymphomas¹², and enforced overexpression of this cluster in hematopoetic stem cells from lymphoma-prone mice accelerated tumor development¹³.

### III. SUMMARY

The invention includes, in one aspect, a method for positively identifying a human miRNA sequence associated with a detectable disease state in humans, such as a cancer. The method includes the steps of (i) identifying, from each of at least two animals having a detectable disease state, such as a cancer, produced by insertional mutation, the sequence of a genomic segment that is common to both animals, and that contains an insertional mutation, (ii) identifying transcription units contained within the animal genome that are within about 200 Kbases, in either an upstream or downstream direction, of the sequenced genomic segment, (iii) identifying human genomic transcription units that are orthologous to the transcription units identified in step (ii), and (iv) for each human transcription unit identified in step (iii), employing a bioinformatics program capable of identifying putative miRNA sequences, to determine whether that transcription unit identified in step (iii) contains a putative miRNA sequence, in which case the putative miRNA sequence is positively identified as a human miRNA.

The detectable disease state may be a cancer, such as lymphoma, wherein step (i) of the method is carried out by isolating the genomic segment from each of at least two animals having a detectable cancer, such as lymphoma. The insertional mutation in step (i) may be a viral insertional mutation.

The sequence identified in step (iii) may be contained in a portion of a pri-miRNA that is outside the corresponding mature miRNA (fully processed miRNA), or it may contained completely within the mature miRNA, or it may be contained in both portions of pri-miRNA transcript.

In another aspect, the invention includes an assay kit for diagnosing the presence or risk of cancer in a human subject. The kit includes a first reagent designed to react specifically with a human pri-miRNA and/or mature miRNA sequence identified in accordance with the method of claim 2, to form a first detectable reaction product, and an indicator guide that indicates how the presence or amount of the reaction product correlates with the presence or risk of the disease state in a human subject.

The first reagent may be one of: (a) PCR reagents for detecting the presence or absence of the genomic sequence, or (b) oligonucleotide binding reagents for detecting the presence of absence of the genomic sequence. For use in diagnosing the presence of risk of a cancer in a human subject, step (i) in the method is carried out by isolating the genomic from each of at least two animals having a detectable cancer, such as a lymphoma. The kit's first reagent may be designed to react specifically with a mature human miRNA sequence identified in accordance with the method of claim 1.

Also disclosed is a method for treating a cancer in a human subject, by administering to the subject, a therapeutically effective amount of a compound capable of binding specifically to a mature human prim-miRNA and/or a mature miRNA sequence identified in accordance with the above method.

Further disclosed is an isolated mature human miRNA sequence selected from the group consisting of SEQ ID NOS: 1-55.

In a more general aspect of the above method, the invention provides a method for identifying a human regulatory RNA (regRNA) sequence associated with a detectable disease state in humans. The method includes the steps of: (i) identifying, from each of at least two animals having a detectable disease state produced by insertional mutation, the sequence of a genomic segment that is common to both animals, and that contains an insertional mutation, (ii) identifying transcription units contained within the animal genome that are within about 200 Kbases, in either an upstream or downstream direction, of the sequenced genomic segment, (iii) identifying human genomic transcription units that are orthologous to the transcription units identified in step (ii), (iv) for each human transcription unit identified in step (iii), using a bioinformatics program to determine whether that transcription unit is a non-coding RNA sequence, and (v) if the homologous human genomic sequence from step (iv) is a non-coding RNA sequence, classifying the sequence as a human regRNA sequence associated with the detectable disease state.

The insertional mutation in step (i) may be a viral insertional mutation. The detectable disease state may be a cancer, wherein step (i) is carried out by isolating the genomic segment from each of at least two animals having a detectable cancer.

The human regRNA sequence may be an miRNA, wherein step (iv) includes employing a bioinformatics program capable of identifying putative miRNA sequences to determine whether that transcription unit identified in step (iii) contains a putative miRNA sequence, in which case the putative miRNA sequence is positively identified as a human miRNA.

The method may further include utilizing the identified human regRNA sequence for diagnostic or therapeutic purposes.

Also disclosed is an assay kit for diagnosing the presence or risk of cancer in a human subject. The kit includes a first reagent designed to react specifically with a human regulatory RNA (regRNA) sequence identified in accordance with the method of claim 15, to form a first detectable reaction product, and an indicator guide that indicates how the presence or amount of the reaction product correlates with the presence or risk of the disease state in a human subject.

As above, the first reagent may be one of: (a) PCR reagents for detecting the presence or absence of the genomic sequence, or (ii) oligonucleotide binding reagents for detecting the presence of absence of the genomic sequence.

In still another aspect, the invention includes a novel regulatory RNA (regRNA), in addition to the novel miRNA identified above, which when overexpressed or disrupted contribute to the formation of tumors. The human and mouse sequences for each regRNA in FASTA format are listed in Table 1B along with the identifying cluster ID. SEQ ID NO:1-55 are mature human miRNAs. SEQ ID NO: 56-110 are mature mouse miRNAs. SEQ ID NO: 111-165 are human pre-miRNAs. SEQ ID NO:166-220 are mouse pre-miRNAs. SEQ ID NO: 221-500 are human pri-miRNAs. SEQ ID NO: 501-822 are mouse pri-miRNAs.

The regRNA disclosed can regulate oncogenes and/or suppressors or actually be an oncogene and/or suppressor itself. The novel regRNA sequences may be used in diagnostic applications, for detecting the presence and/or risk of a given cancer type, or in therapeutics, e.g., for treating that cancer

These and other aspects, objects, advantages, and features of the invention will become apparent to those persons skilled in the art upon reading the details of the invention as more fully described below.

### IV. BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIGS. 1A and 1B are customized screen prints of the UCSC genome web site browser (March 2005 version of the mm6 gene assembly), looking at the mir-17-20 locus (FIG. 1A); and at the mir-106a-92 locus (FIG. 1B). Mir-17-20 is the mouse cluster orthologous to the human mir-17-92 cluster. Mir-19b-1 only weakly maps to the mouse genome at the indicated location. Top, base position at chromosomes 14 and X, respectively. The handle bars below “Picobella_SL3” represent the retroviral insertions into the mir-17-20 locus (FIG. 1A) or the mir-106a-92 locus (FIG. 1B) in 29 or in 33 independent tumors, respectively. The bars below “miRNA”, are miRNAs found in the miRNA registry 10 (//www.sanger.ac.ukl/Software/Rfam/mirna/); the bars below “miRNA predicted” represent miRNAs predicted by use of the method herein. The exon/intron structure of mRNAs and ESTs of the mouse is shown below the predicted miRNA. Sequence conservation between mouse and various other species (rat, human, dog, cow, opossum, chicken, tropicalis, zebrafish, and tetraodon) is also shown.

FIGS. 2A and 2B are each a customized screen print of the UCSC genome web site browser, looking at two loci with predicted miRNA located on chromosomes 8 and 12, (FIGS. 2A and 2B, respectively). For FIG. 2A, the two handle bars below “Picobella_SL3” (1490S-206-1 and 1163S-137-14), represent retroviral insertions into the locus recovered in 2 independent tumors. Known miRNAs listed in the miRNAs registry 10 are not found in this locus; the 2 bars below “miRNA predicted” represent miRNAs predicted by use of the method herein. Two retroviral integrations (S3_{—}306D and S5_{—}030A1) represent independent tumors as listed in the RTCGD database 14 (Retrovirus Tagged Cancer Gene Database; //RTCGD.ncifcrf.gov). In FIG. 2B, the handle bars under “Picobella_SL3” represent retroviral insertions into the locus recovered in 8 independent tumors. The bars for “miRNA predicted” are miRNAs predicted by the method herein. Known miRNAs listed in the miRNAs registry 10 are not found in this locus. The AK019999, AI1060616, BE848409, and BB634791 transcripts are thymus-specific. Sequence conservation between mouse and various other species is also shown.

FIGS. 3A and 3B are each a customized screen print of the UCSC genome web site browser, looking at two loci with regulatory RNA. The top of the figures shows the base position at chromosomes 15 and 1 (FIGS. 3A and 3B, respectively). The handle bars below “Picobella_SL3” represent the retroviral insertions recovered by the present method in 7 independent tumors (chr. 15, FIG. 3A); and 5 independent tumors (chr 1, FIG. 3B). Arrows within handle bars denote transcriptional direction. The exon/intron structure of mRNAs and ESTs of the mouse are shown below the predicted miRNAs. Transcripts AK040104 and AK041852 (FIG. 3A) and BY097680 (FIG. 3B) are thymus-specific. Sequence conservation between mouse and various other species is shown at the bottom.

FIG. 4A is a table showing tumors assayed for the region containing mmu-mir-106a (FIG. 1B). Retroviral insertion site locations (August 2005 version of the mm7 genome assembly) are notated by the basepair located directly after the insertion. Orientation of the retrovirus is indicated by “+++” for directionality of left to right and by “−−−” for directionality of right to left on the chromosome.

FIG. 4B is a graph of the relative expression of AY940616 as measured by quantitative PCR. Tumors with integrations located upstream of AY940616 (the predicted primary transcript for the mmu-mir-106a-92 locus) were assayed by qPCR using a dual labeled probe designed to AY940616. Integration sites assayed were located within (i) ˜3 kb, (ii) ˜14 kb, and (iii) ˜18 kb upstream of AY940616. Tumors with no integrations in this region (iv) along with cDNA from a normal mouse spleen were run as controls. Beta-actin (ACTB) was used as the endogenous reference gene and 1735S, one of the tumor controls, was used as the calibrator sample in the calculation of 2^{-ΔΔCt} values. All 2^{-ΔΔCt} values were normalized such that the average of the tumor controls was set to 1.

FIG. 4C is a graph of the relative expression levels of mmu-mir-106a by quantitative PCR. Tumors with integrations located upstream of the mmu-mir-106a-92 locus were assayed by qPCR using a reverse transcriptase primer/dual labeled probe system designed to mmu-mir-106a. Integration sites assayed were located within (i) ˜3 kb, (ii) ˜14 kb, and (iii) ˜18 kb upstream of the miRNA cluster. Tumors with no integrations (iv) in this region were run as controls. Concentrations of mmu-mir-106a were determined using a standards curve generated with a synthetic mmu-mir-106a RNA oligo. Concentrations were then normalized by the average of the tumor controls to calculate relative expression levels.

FIG. 5A is a map of the region containing AK030859. The genomic organization of retroviral insertion sites in the region containing AK030859 is shown by a screen capture of the UCSC genome website browser (August 2005 version of the mm7 genome assembly). Insertion sites are drawn as vertical handlebars below “PicoSL3”.

FIG. 5B is a table showing tumors assayed for the region containing AK030859. Tumor locations and orientations are notated as in FIG. 4A.

FIG. 5C is a graph showing the relative expression of AK030859 as measured by quantitative PCR. Tumors with integrations located in the region encompassing AK030859 were assayed by SYBR qPCR for the 5′ end of AK030859. Integration sites assayed were located (i) up to 1.2 kb upstream, (ii) within, and (iii) up to 52 kb downstream of AK030859. Tumors with no integrations in this region (iv) were run as controls. Beta-actin (ACTB) was used as the endogenous reference gene and 1484S, one of the tumor controls, was used as the calibrator sample in the calculation of 2^{-ΔΔCt} values. All 2^{-ΔΔCt} values were normalized such that the average of the tumor controls was set to 1.

FIG. 6A is a map of region containing AK040062. The genomic organization of retroviral insertion sites in the region containing AK040062 is shown by a screen capture of the UCSC genome website browser (August 2005 version of the mm7 genome assembly). Insertion sites are drawn as vertical handlebars below “PicoSL3”.

FIG. 6B is a table showing the tumors assayed for the region containing AK040062. Tumor locations and orientations are notated as in FIG. 4A.

FIG. 6C is a graph showing the relative expression of AK040062 exon 2 as measured by quantitative PCR. Tumors with integrations located in the region encompassing AK040062 were assayed by SYBR qPCR for AK040062 exon 2. Integration sites assayed were located (i) up to 6 kb upstream, (ii) within intron 1, (iii) within intron 2, and (iv) up to 16 kb downstream of AK040062. Tumors with no integrations in this region (v) along with normal mouse spleen samples (vi) were run as controls. Data was treated as previously mentioned for AK030859 except 3412S was used at the calibrator sample.

FIG. 7A is a map of the region containing AK037419. The genomic organization of retroviral insertion sites in the region containing AK037419 is shown by a screen capture of the UCSC genome website browser (August 2005 version of the mm7 genome assembly). Insertion sites are drawn as vertical handlebars below “PicoSL3”.

FIG. 7B is a table showing the tumors assayed for the region containing AK037419. Tumor locations and orientations are notated as in FIG. 4A.

FIG. 7C is a graph showing the relative expression of AK037419 exon3 as measured by quantitative PCR. Tumors with integrations located in the region encompassing AK037419 were assayed by SYBR qPCR for AK037419 exon 3. Integration sites assayed were located (i) up to 13 kb upstream, (ii) within intron 1, (iii) within intron 2, and (iv) within exon 3 of AK037419. Tumors with no integrations in this region (v) along with normal mouse spleen and thymus samples (vi) were run as controls. Data was treated as previously mentioned for AK030859 except 1438S was used as the calibrator sample.

FIG. 8 is a graph showing relative expression of PVT1 exon 1 in matched human normal and tumor prostate RNA samples. Matched human normal and tumor prostate RNA samples were assayed by SYBR qPCR for PVT1 exon 1. Beta-actin (ACTB) was used as the endogenous reference gene and each normal RNA was used as a calibrator for its matched tumor RNA in calculating 2^{-ΔΔCt} values.

Table 1A includes a seven page list of regulatory RNA clusters. Tumors with proviral integrations, representative ESTs, and known and predicted miRNAs found at each loci are indicated. Chromosomal locations are from version mm6 of the mouse genome and the hg17 version of the human genome at the UCSC Genome Bioinformatics website (genome.ucsc.edu). “Known miRNAs” refers to miRNAs found in the miRNA registry (August 2005); “Predicted miRNAs” refers to miRNAs predicted as described in the text. Since the miRNA cluster mir-17-92 has been previously described as a possible oncogene 13, the mir-17-20 and mir-17-92 sequences are not included in Tables 1B. The human and mouse sequences for each regRNA in FASTA format are listed in Table 1B along with the identifying cluster ID. SEQ ID NO:1-55 are mature human miRNAs. SEQ ID NO: 56-110 are mature mouse miRNAs. SEQ ID NO: 111-165 are human pre-miRNAs. SEQ ID NO:166-220 are mouse pre-miRNAs. SEQ ID NO: 221-500 are human regRNAs. SEQ ID NO: 501-822 are mouse regRNAs. SEQ ID NO: 14, 26, 37-39, 41-43 are known human miRNAs that were not previously known to be associated with cancer.

Tables 2A and 2B are two and three page lists, respectively, of miRNAs, regRNA, ESTs, or genes, co-mutated with the mir-17-20 locus (Table 2A) or the mir-106a-92 locus (2B). The predicted miRNAs are in bold. Co-mutated regions in common between the mir-17-20 and the mir-106a-92 loci are indicated by asterisks (**). Chromosomal locations are from version mm6 of the mouse genome at the UCSC Genome Bioinformatics website (genome.ucsc.edu). “Indeterminate” refers to regions where the miRNA, EST, or gene could not be determined. “Desert” regions are those which appear to be void of miRNAs, ESTs, or genes.

### V. DETAILED DESCRIPTION

### A. Definitions

The following terms have the definitions given below, unless otherwise indicated in the specification.

“Regulatory RNA” or “regRNA” generally refers to non-protein encoding RNA molecules (including miRNA) that regulate the expression of genes.

“microRNA” or “miRNA” generally refer to ˜18-24-mer RNAs that regulate the expression of genes by binding to the 3′-untranslated regions (3′-UTR) of specific mRNAs. According to standard nomenclature, a pre-processed miRNA transcript prior is referred to an pri-miRNA. Enzymatic cleavage of pri-miRNA in the nuclear compartment by Drosha yields a pre-miRNA, which is further processed by Dicer in the cytoplasmic compartment in form mature miRNA. “miRNA” may be used herein to refer to pri-miRNA, pre-miRNA or mature miRNA, and the distinction, if any, will be understood from the context in which it is used.

“Stringent conditions” refers to a procedure including a stringent wash such as with 0.1% saline sodium citrate, and 0.1% sodium dodecyl sulfate (0.1% SSC, 0.1% SDS) at 65° C. Appropriate stringent conditions are further described in Sambrook et al., Molecular Cloning, Cold Spring Harbor Laboratory Press, New York, 1989.

As used herein, a nucleotide or RNA sequence “specifically hybridizes” to a sequence under physiological conditions, with a Tm substantially greater than 37° C., preferably at least 50° C., and typically 60° C., 80° C. or higher. Such hybridization preferably corresponds to stringent hybridization conditions, selected to be about 10° C., and preferably about 50° C. lower than the thermal melting point (T[m]) for the specific sequence at a defined ionic strength and pH. At a given ionic strength and pH, the T[m] is the temperature at which 50% of a target sequence hybridizes to a complementary polynucleotide.

Polynucleotides are described as “complementary” to one another when hybridization occurs in an antiparallel configuration between two single-stranded sequences. Complementarity (the degree that one polynucleotide is complementary with another) is quantifiable in terms of the proportion of bases in opposing strands that are expected to form hydrogen bonds with each other, according to generally accepted base-pairing rules.

The term “overexpressed” refers to a range of expression of a protein which is greater than that generally observed for a given type of cells.

The term “insertional mutation” refers to a mutation that is introduced into a genome by insertion of an exogenous sequence or an endogenous sequence. Such exogenous and endogenous sequences may be, for example, either viral or transposon-based. An insertional mutation may enhance the transcription of one or more coding or non-coding genes located within about 200 Kbases of the mutation.

The term “orthologous sequence” refers to a sequence having a direct evolutionary counterpart derived from a common ancestor by vertical descent; and, as a consequence, having conserved function to a high degree of likelihood.

A “bioinformatics program” refers to computer program designed to carry out one or more sequence analysis functions on database sequences. These functions may include sequence alignment, recognition of regions capable of forming secondary structure, recognition of various gene transcription and/or translation control sequences, and identification of one or many possible different classes of genomic sequences, including coding sequences in general, and coding sequences for particular types of proteins, non-coding gene sequences, transcription splice sites, secondary structure sites, identification of genes for various cellular RNAs, and recognition of orthologous genes from different organisms.

A “transcriptional unit” refers to a coding or non-coding gene, or the transcript produced thereby, and may be identified, for example, by the presence of a polyadenylation site on the corresponding processed transcript.

### B. Methods of Identifying regRNA

Regulatory RNA and miRNA sequences that contribute to tumor formation are described and disclosed. These regRNA and miRNA sequences were identified in mice, and were subsequently confirmed in humans. These sequences were identified by the following methods.

A retrovirus that induces tumors was used to identify 322 loci encoding regRNAs, many of which are expressed only in thymocytes. Of these loci, 29 are predicted by current algorithms to encode miRNA, and four are confirmed miRNA polycistrons listed in the miRNA registry. miRNA overexpression was confirmed for several tumors containing nearby integration sites predicted to activate transcription. These results (a) substantially increase the number of known miRNAs and (b) identify them as being oncogenic when dysregulated in T cells.

Because the expression of a large number of miRNAs is dysregulated in lymphomas^{8,9}, it seemed likely that many more miRNAs than were previously known act as oncogenes or tumor suppressor genes. The present method defines oncogenic miRNAs and other regRNAs in a high throughput manner using proviral tagging. Although viruses have not yet been implicated as a major cause of cancers in humans, research using tumor viruses has led to the discovery of many oncogenes and protooncogenes. In proviral tagging methods, mice are infected with a retrovirus that does not contain an oncogene (e.g., murine leukemia virus, MLV, or murine mammary tumor virus, MMTV). Recently, the host range of this approach has been broadened by the use of a transposon^{15,16}.

During retroviral infection, the virus integrates into the cellular genome and inserts its DNA near or within genes, which leads to various outcomes:

(i) The insertion site is too far away from a protooncogene and thus does not activate it. In this case, there will be no selection for that cell.

(ii) The provirus inserts within 200 kb of a protooncogene, but not within the gene (type 1). Here, either the viral promoter or the viral enhancer increases the expression level of the protooncogene.

(iii) The provirus inserts within a gene, destroying or altering its function (type 2).

There will be no selection for a cell that contains either type 1 or type 2 insertion events in a gene that is not a protooncogene or tumor suppressor gene. If integration results in the formation of a tumor, genes adjacent to the integration site can be identified, and classified as either protooncogenes or tumor suppressor genes. This method has been used to identify many new protooncogenes as well as to confirm already known protooncogenes discovered by virtue of their homology to viral oncogenes¹⁷⁻²⁵. A tumor suppressor may be scored if a retrovirus lands within a gene and truncates or destroys it. In these cases, the suppressor may be haplo-insufficient, or alternatively, the mutation on the other allele is provided spontaneously by the mouse. The integration event may also lead to more complex consequences, such as a dominant negative effect of the truncated gene product or the transcription of anti-sense or miRNA.

Because the mechanics of transcription of pri-miRNA and regular nuclear gene transcripts are the same, it was reasoned that retroviral insertions near or into these transcribed regions ought to have similar effects. Whereas to date, all mammalian miRNAs have been discovered by computational methods, the present methods provide an extensive forward genetic approach to functionally identify novel oncogenic miRNAs in retrovirally generated tumors.

The present invention, in one non-limiting embodiment, provides a method of identifying novel human regulatory RNA (regRNA) sequences, including novel miRNA sequences, associated with a detectable disease state in humans. In practicing the method, an animal model, such as mouse or rat, having known disease states, and typically disease states that are similar to those found in humans, is subject to standard insertional mutagens, such as viral insertional mutagens, and then observed for development of one or more disease states, e.g., one or more cancer types, or hyperlipidemia, both diseases known to be associated with dysfunctions in regRNA. When a disease state is observed in a mutagenized animal, the genome, e.g., in a cancerous tissue or cell, is then analyzed for the presence and chromosomal locations of the one or more insertion mutations. This is done, for example, using PCR probes that overlap with the insertional mutagen sequence, to produce an amplified segment of the animal genome adjacent the mutation.

The sequence of this segment is then determined and used in a database search of the animal's genome, to find transcriptional units that are within a defined distance, typically less than 100 Kbases, but up to 200 Kbases, upstream and/or downstream of the insertional mutation site or sequenced segment containing that site. Transcriptional units are identified according to known procedures, e.g., by employing a bioinformatics program that stores information about transcription units that have been previously identified as such by the presence of polyadenylation in their transcripts.

For each transcriptional unit that is identified in this manner, the method now involves searching a human genomic database to identify human transcriptional units that are orthologous with the identified animal transcription units. This step is used in finding the human transcription unit corresponding to the one identified in the animal as possibly related to an identified disease state. Of course, since some animal transcription units are unique to that animal and/or do not overlap with human transcription units, not every animal transcription unit identified in the method will have a human ortholog.

Once the human transcription units corresponding to the disease-related animal transcription units have been identified, these are further analyzed using bioinformatics tools to (i) identify those non-coding units that will be classed as regRNAs, and (ii) among the regRNAs, those units that contain secondary structure and other sequence-related features associated with miRNAs. In the first case, a human transcription unit identified as above is compared against known coding sequences, or sequences with coding-gene sequence features, to determine whether the transcription unit is a coding or non-coding gene. If it is a non-coding gene, and not previously identified as a regRNA sequence, or not previously identified as having the newly identified disease association, the method identifies the transcription unit either as a novel regRNA, or a known regRNA having a newly-identified disease associated function.

If the regRNA is further determined to contain sequences characteristic of miRNAs, e.g., stem-loop regions characteristic of pre-miRNAs, then the method can further identify the regRNA as either a newly identified miRNA sequence (including the pri-miRNA, the pre-miRNA, and/or mature miRNA), or a previously known miRNA with a newly identified disease association (SEQ ID NOS: 13, 14, 26, 27, 37-39, and 41-43.)

It will be appreciated that the method just described, which combines a functional assay (disease association) with a bioinformatics analysis, allows confirmation or positive identification bioinformatics information, e.g., gene identification, and also allows for less stringent bioinformatics constraints, e.g., in the identification of novel miRNAs, as discussed below.

Also forming part of the invention are a comprehensive set of regRNAs including miRNAs that when overexpressed or deleted contribute to tumor formation. The miRNAs can regulate both oncogenes and suppressors, as well as represent both oncogenes and suppressors themselves. Although classic tumor suppressors require both alleles to be inactive, the present recovery of regRNA sequences used a modified retroviral tagging strategy. In this modified strategy, chemical mutagenesis was initially carried out on the paternal allele, followed by retroviral insertional mutagenesis (which can affect both the maternal and paternal alleles). Chemical mutagenesis was carried out using ENU (N-ethyl-N-nitrosourea; a potent germ line mutagen). If by chance the virus-disrupted (maternal) allele and the ENU-inactivated (paternal) allele belong to the same locus, then the cell has no functional allele. Should this locus represent a tumor suppressor, the cell lacking it will have a growth advantage over other cells, which may result in tumor formation.

1. Viral Tag Recovery and Locus Identification

The viral integrations sites (tags) were determined in tumors generally by isolating and digesting genomic tumor DNA, followed by an anchored PCR technique 20. This was performed by amplifying and sequencing a chimeric DNA fragment consisting of a short genomic sequence upstream of the viral 5′ LTR and part of the viral 5′ LTR itself. The tags were sequenced and mapped to the mouse genome sequence, and the affected transcription unit was determined. From 2373 tumors, 7300 tags were obtained, which mapped to 2,038 regions. Of these regions, 645 had two or more associated integration sites, with the largest region having 500 integrations.

2. Calling Regulatory RNA Transcripts

At least one of the following, non-limiting, considerations should be taken into account to correctly identify the affected regRNA based on the retroviral screen. First, although vertebrates share extensive highly conserved non-coding sequences which might represent regRNA, not all non-translated (translatable) expressed sequence tags (ESTs) fragments represent true regulatory RNA. For example, a fraction represent small nuclear RNA of the spliceosome, another fraction results from DNA contamination, and yet another fraction may just be transcriptional noise not yet edited by evolution for energy efficiency. Second, viral integration into a potentially transcribed region does not necessarily mean this transcription unit is activated and contributes to tumorigenesis. There is the question whether or not the proviral enhancer/promoter can “leapfrog” the nearest gene and instead regulate the next one. In the past, it has been assumed that this is not (or only rarely) the case, and that proviruses can exert their function up to a distance of 200 kb from a gene. Such assumptions were reevaluated in light of more extensive genomic coverage and better annotation of non-coding transcripts. With the above potential complications in mind, the transcription unit nearest to a cluster of integration sites was identified. In the analysis, it was reasoned that if a gene is located, for example, 200 kb from an insertion site, then the other integration sites ought to be more or less evenly distributed over that distance. If, however, a cluster of integration sites spans a few kilobases and is located within or next to a noncoding transcription unit, this unit was called rather than a far away gene.

3. miRNA Identification

Early computational algorithms designed to predict miRNAs relied on sequence conservation between species, hairpin structure determination, and thermodynamic stability. A more recent prediction attempt has relaxed the species conservation requirement in an attempt to identify new primate-specific miRNAs¹¹. Nonetheless, all computational approaches involve a trade off between maximizing sensitivity and minimizing false positives, and as such, may miss important classes of miRNAs. Since the retroviral screen provided complementary functional data, it was possible to modify the computational approach of Berezikov et al.²⁶ with relaxed input parameters and maintaining the sequence conservation between mouse and human as a necessary condition. This computational approach yielded 13,648 predicted miRNAs. Apart from non-translatability, ESTs terminating at the 3′ or 5′ end of the miRNA cluster were identified, which should be an indication for a site of Drosha processing activity. Based on these criteria, retroviral integrations at 322 loci with regRNAs were found, many of which are expressed only in thymocytes. These include integrations at: (1) mir-17-20, the mouse ortholog to the human miRNA cluster (mir-17-92) that has been demonstrated to be an oncogene in mouse and likely in humans¹³; (2) three other confirmed miRNAs in the registry; (3) 29 non-coding transcription units with predicted miRNA; and (4) 289 non-coding transcription units without miRNA predicted.

Table 1A is a list of the 322 mouse and 280 human regRNA and miRNA loci. For each cluster, the cluster ID, the chromosomal location, the tumors that contain the proviral integrations sites in that cluster, the ESTs within and adjacent to that cluster, the known and predicted miRNAs, and the genomic location of the corresponding human regRNA are listed. The chromosomal positions of the mouse regRNA and miRNAs are defined by the March 2005 UCSC genome assembly of the mouse genome (mm6) while the chromosomal positions of the human regRNA and miRNAs are defined by the hg17 UCSC human genome assembly. The sequences of the regRNA and miRNAs are listed in Table 1B in FASTA format, with the exception of the mir-17-20 and mir-17-92 loci. Examples of the groups are disclosed and described below.

4. mir-17-20 and mir-106a-92

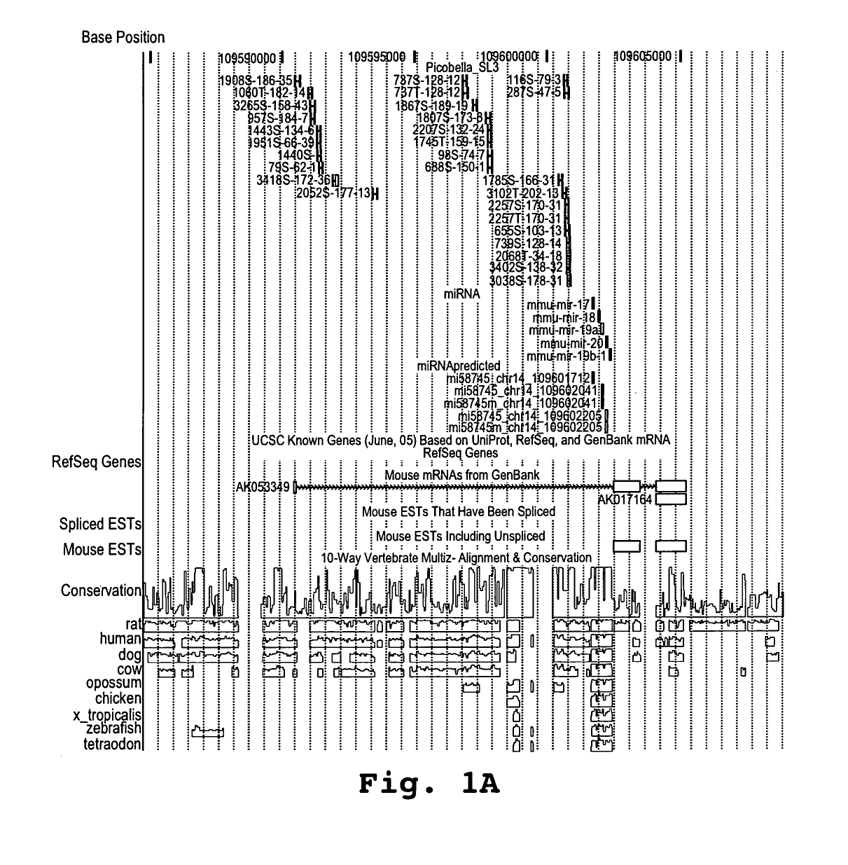

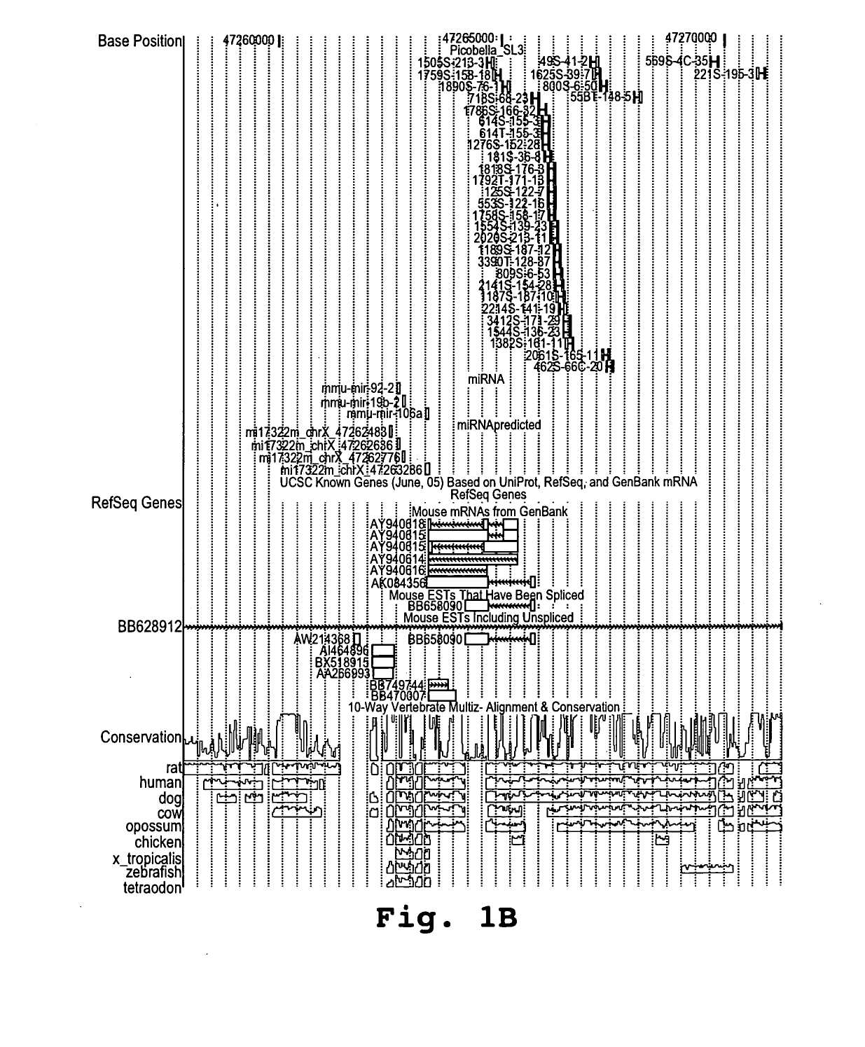

The mir-17-20 polycistron contains four confirmed miRNAs, three of which are predicted by the bioinformatics approach of the present method (FIG. 1A; mir-19b-1 only weakly maps to this cluster). To date, this polycistron is the only one that has been shown to be an oncogene in the mouse¹³. Several of the ESTs terminate 3′ of the cluster and all 5 miRNAs are contained in the intron of transcript AK053349. The 29 retroviral insertion sites fall into three groups, all contained in the kb transcription unit. It is unclear why there are these three groups, but perhaps site specificity of Drosha or undetected novel miRNAs are the cause. Interestingly, all 11 integrations closest to the mir-17-20 polycistron have the same direction (left to right) of transcription as the miRNAs themselves (left to right; not shown). Conversely, 9 out of 10 of the integrations farthest from the polycistron have the opposite orientation (right to left) of the miRNAs. The orientation of the provirus is thought to be important in activation of protooncogenes. Either the viral promoter, in the same transcriptional orientation as the protooncogene, overrides the promoter of the protooncogene, or the enhancer, in either orientation, cooperates with the promoter to increase transcription of the cellular gene. In the classical insertions of type 2, i.e., within a gene, the result is either truncation or destruction. Because mir-17-92 polycistron acts as an oncogene 13, it ought to be the case that 3′ to the integration sites there are transcripts generated at an increased level, and that these transcripts can be processed by Drosha and Dicer.

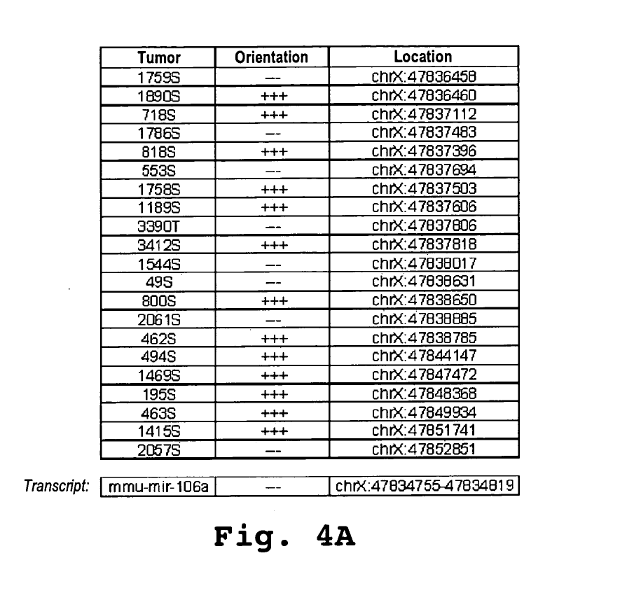

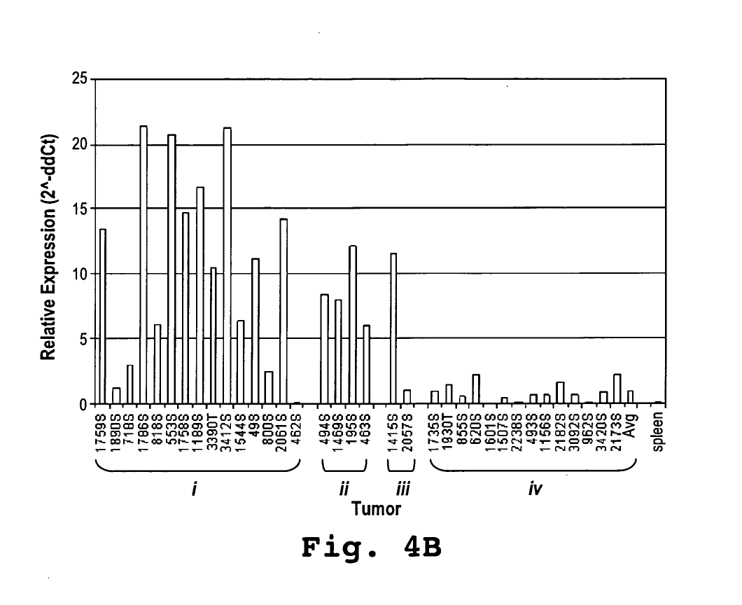

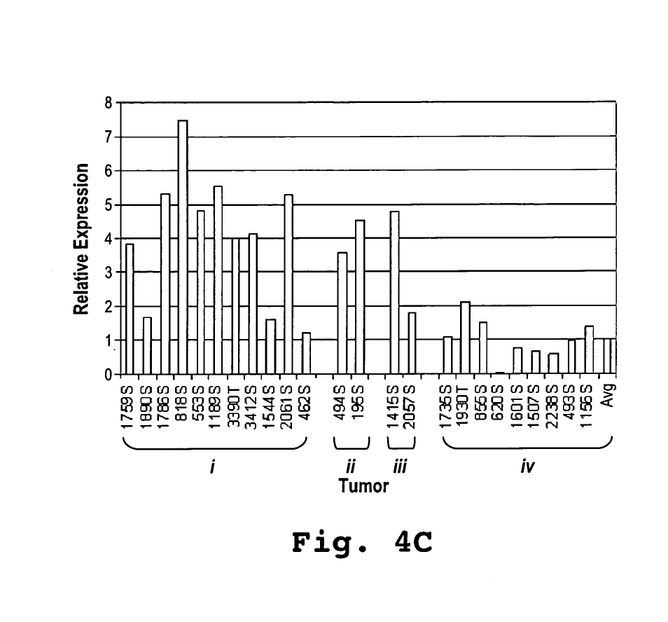

The mir-106a-92 polycistron is a cluster related by homology to mir-17-92²⁷ and contains three previously identified miRNAs and one more predicted by us (FIG. 1B). The transcript AK084356 ends precisely where the miRNA cluster begins, and part of the intron is an exon of other transcripts. There are also several more near the miRNA cluster. The two leftmost proviral integrations (1505S, 1759S) have the same transcriptional orientation as the AK084356 transcript and thus may constitute “promoter insertions”. Because of their distance to the transcription unit, the 3 rightmost retroviral insertions (558T, 569S, 2221S) ought to represent enhancer insertions. In these cases, the provirus has integrated 5′ to a transcription unit, and the orientation of transcription of provirus and cellular transcription unit are opposite. This is because in the LTR of the provirus, the enhancer precedes the promoter and it is thought that the enhancer cooperates with promoters without leapfrogging. The remaining integrations may be either promoter or enhancer insertions and thus may have either orientation. Transcript AY940616 and mature the mmu-mir-106a miRNA were both found to be overexpressed in mouse thymic tumors by quantitative PCR (FIG. 4A-C).

5. Oncogenic miRNAs Not Found in the miRNA Registry

The number of existing miRNAs is growing monthly. In early 2005, the number in humans was roughly 200, and early estimates calculated 255 as the upper limit²⁸. There are 321 human miRNAs in the most recent version of the miRNA registry (August 2005). Recent studies have suggested that the number of human miRNAs may be much greater, and as much as 800²⁹.

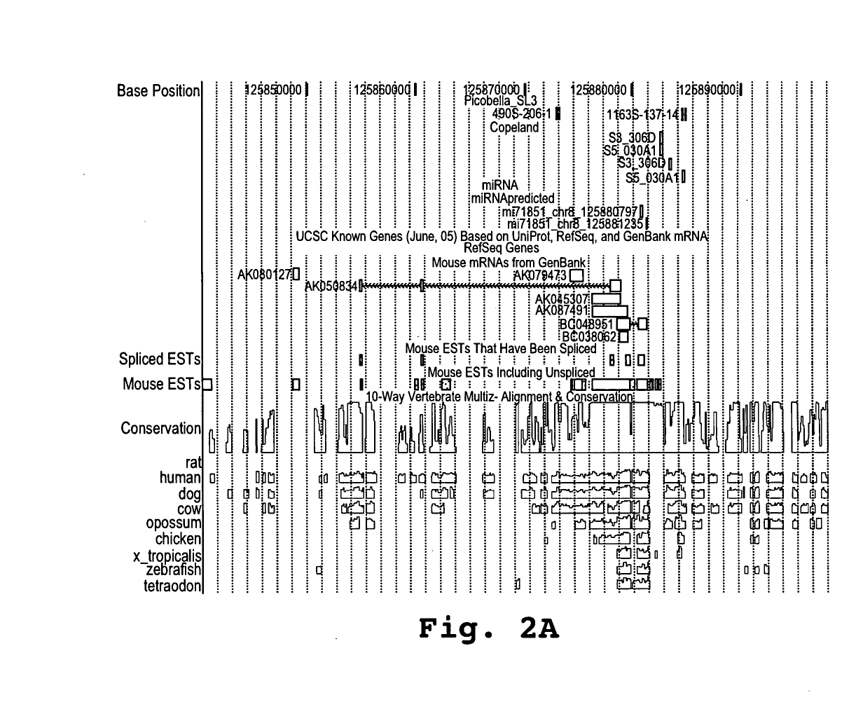

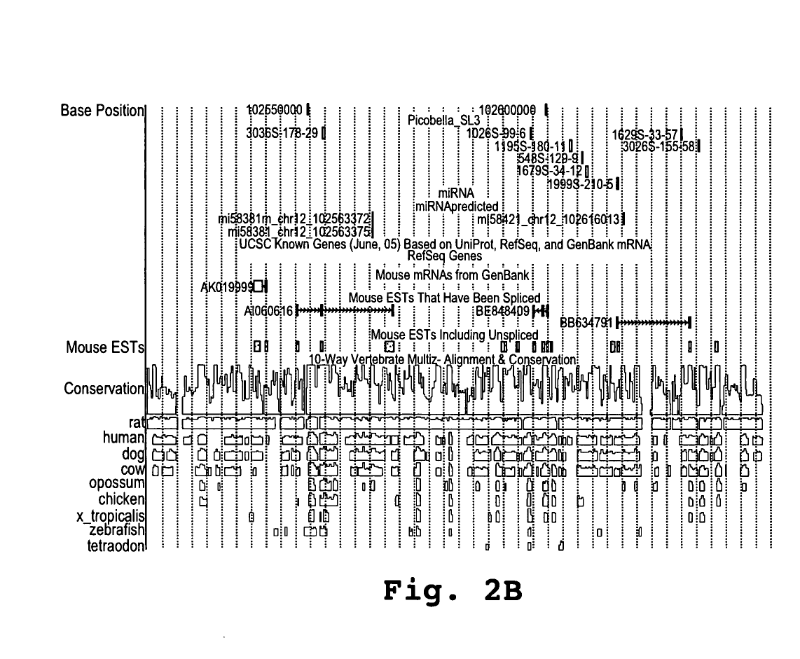

As seen in FIG. 2A the predicted miRNAs are contained in transcript BC048951, and are close to other ESTs that may be processing products of Drosha and Dicer. Thus, part of transcripts AK045307 and AK087491 overlap with BC048951, and another part is contained in the intron of the much longer transcript AK050834. An additional transcript that covers part of the same intron is thymus specific AK079473. The retroviral insertion site 1490S is within the large intron of the AK050834 transcript, which presumably represents the largest piece of the pri-miRNA. The other insertion, 1163S, is 3′ to the pri-miRNA, in the same transcriptional orientation, which allows the viral enhancer to cooperate with the promoter of the pri-miRNA.

FIG. 2B shows 8 insertions near two predicted mi-RNAs. Each miRNA is contained in a transcript that is found only in thymocytes (A1060616, BB634791). Interestingly, two other nearby transcripts are also found only in thymocytes.

The prediction program described herein was shown to find 81% of all registered miRNA in the mouse. There are other programs that compare regulatory motifs in promoters and 3′ UTRs in several mammals²⁹. The method also found many regions where no miRNA was predicted, but where the retroviral insertions were (1) within or nearby a transcript that was not translatable and (2) were often far away (>30 kb) from any other gene.

6. Retroviral Insertions into regRNA without miRNAs

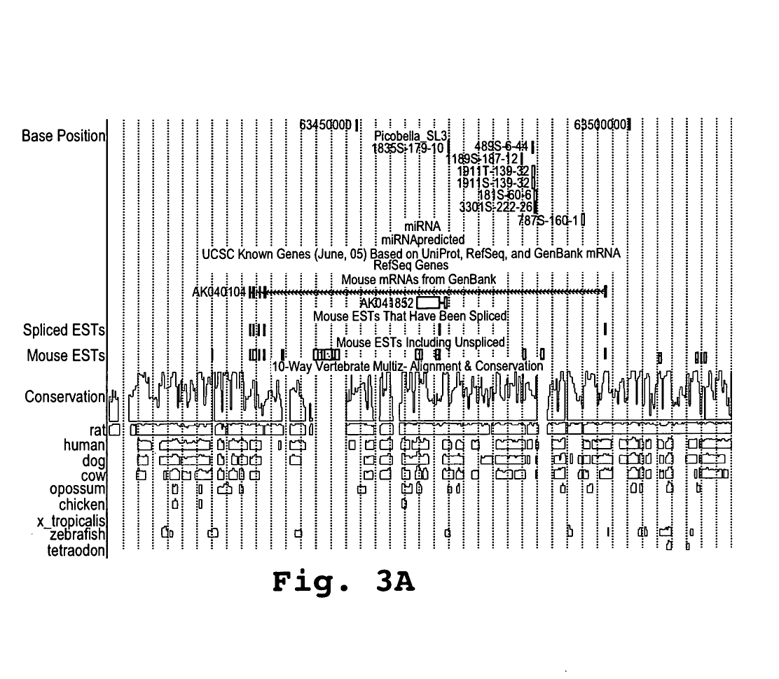

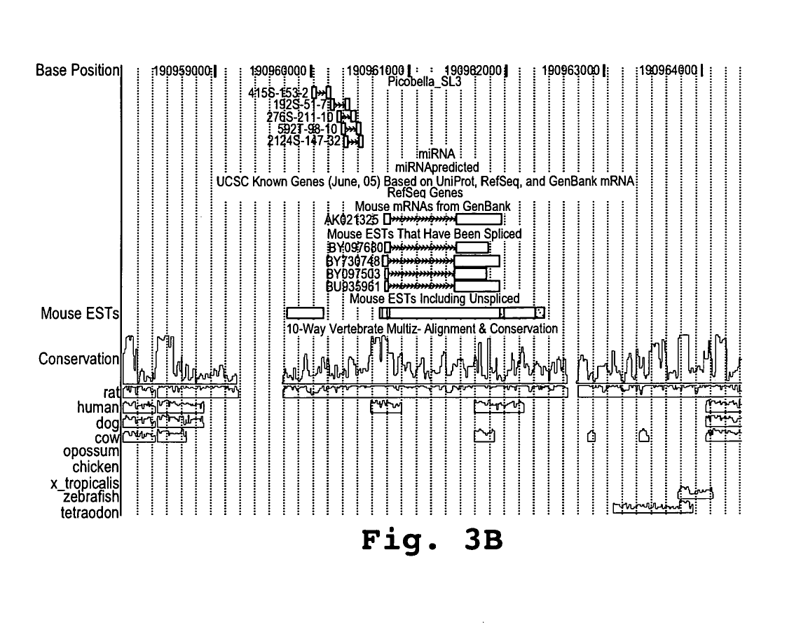

The transcript AK040104 in FIG. 3A, for example, with eight proviral insertions sites, looks like a gene, except that it is not classifiable and is >300 kb away from the nearest known gene. There is a smaller transcript AK041852, which covers two introns of the larger transcript, and both transcripts are expressed only in thymocytes.

FIG. 3B shows 5 integration sites upstream of transcript AK021325 which also lack predicted miRNAs and is ˜40 kb away from the nearest authentic gene. All 5 integration sites have the same direction of transcription as the ESTs, suggesting that transcription of these ESTs in increased by the viral promoter. Thus, insertions into these types of regions were also surveyed, where there was a hint of Drosha processing activity and where thymocyte-specific expression is observed. These regions contain regulatory RNAs, resulting in identification of 289 new regions.

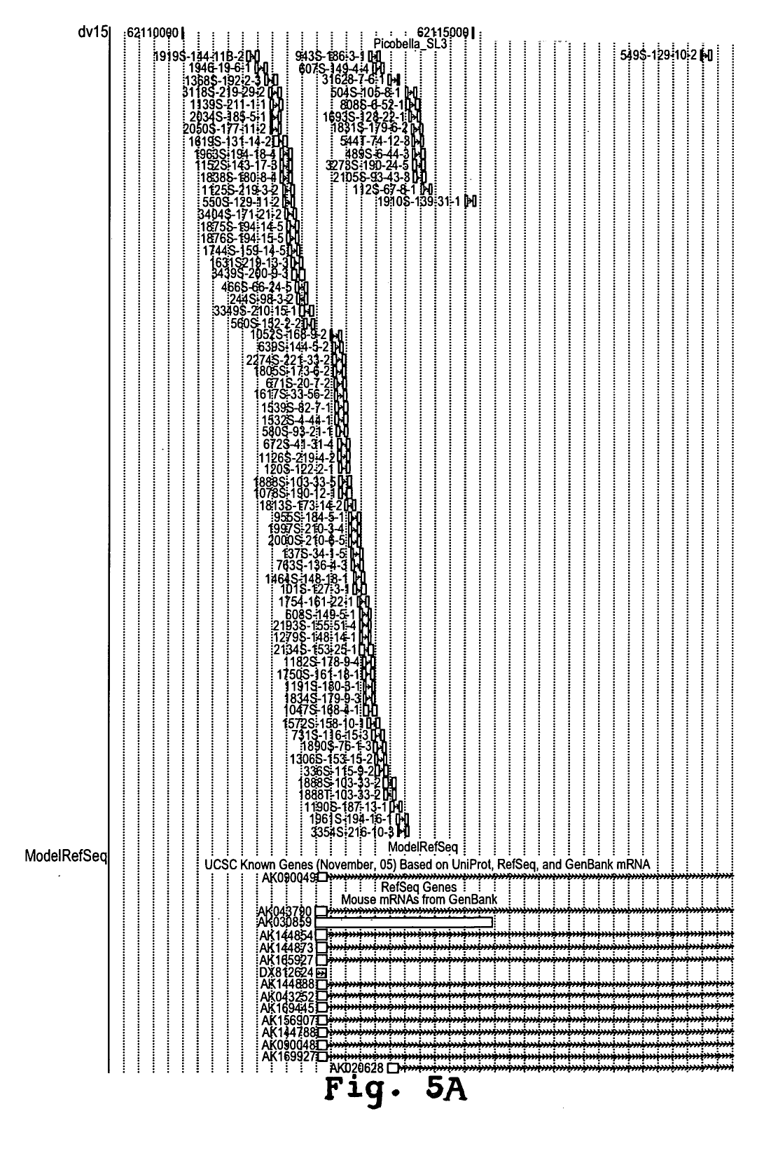

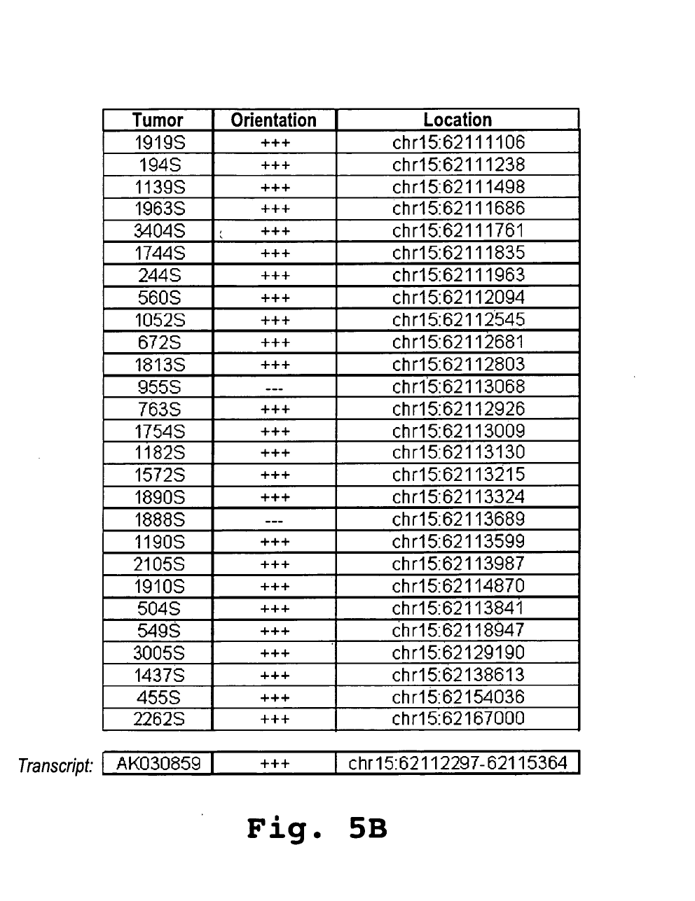

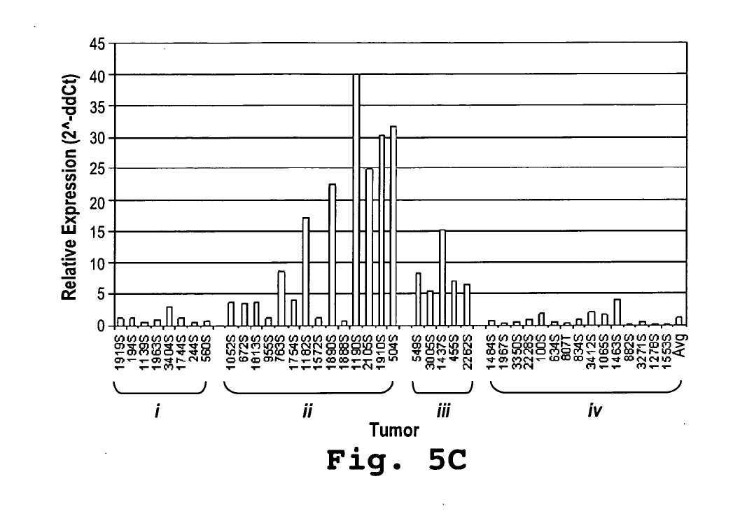

FIGS. 5-7 show three additional loci containing retroviral integrations near or within non-coding regRNAs. The expression levels of each regRNA were measured using quantitative methods; each of these regRNAs was found to be overexpressed in the majority mouse thymic tumors containing nearby integrations as compared control tumors that lacked such integrations.

7. Expression Levels of regRNAs and miRNA in Human Tumors

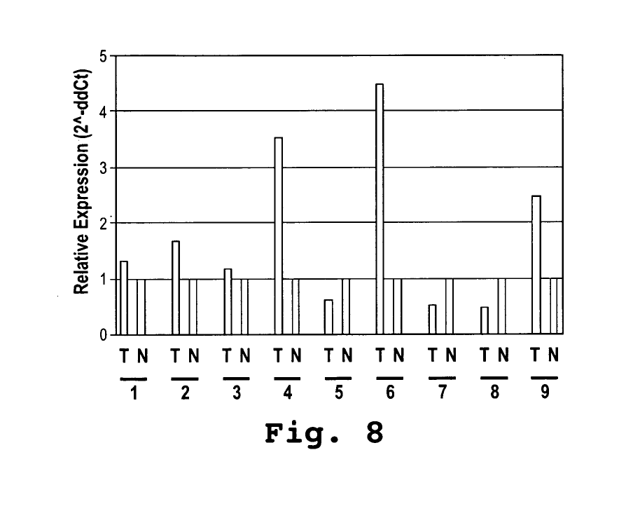

The RNA expression level of a newly identified regRNA (PVT1) was measured in human tumors using quantitative methods (FIG. 8). In 3 out of 9 tumors, expression levels of the specific regRNA were elevated as compared to the level in matched normal tissue from the same patient. The change in expression levels may indicate how regRNAs and miRNAs can be used for diagnosis and therapy of the respective tumors for those skilled in the art.

8. Multistep Tumorigenesis and Co-Mutation Analysis

Co-mutation analysis may be a powerful way to find cooperating signaling pathways in tumorigenesis. Viral insertional mutagenesis, while perhaps not providing all the mutations necessary for a full-blown tumor, follows the multistep scenario of spontaneous tumorigenesis. Lymphocytic tumors that arise as a consequence of infection with MLV can contain up to 7 insertion sites. This fact can be used to differentiate between signaling pathways within a tumor: because multiple oncogenic hits along a signaling pathway may not be selected over a single hit, the genes actually recovered are likely not to be involved in the same pathway, but in complementary pathways that work together in tumorigenesis.

There are generally, however, two main caveats when considering co-mutation analysis. First, although in general, almost all viral insertions in a tumor are thought to be causative in its formation, the question arises whether there are any “passenger” insertions, i.e., insertional events that are not selected by tumorigenesis, but merely accompany other causative mutations. Passenger insertions do not seem to occur frequently due to the superinfection barrier and because secondary integration events are rare. These rare events, however, are responsible for the tumor formation by retroviruses. It is not clear whether the additional insertions are generated by re-infections or by retrotransposition. At any rate, even though passenger mutations have not yet been identified in previous studies, one needs to guard against interpreting such insertions as tumorigenic events—especially when the screen is large. The second confounding issue may be the potential oligoclonality of tumors. If the tumors are not clonal, then what is scored as a co-mutation may simply be a mutation in a different tumor.

With these caveats, co-mutation analysis provides valuable insight into the pathways that work together during tumorigenesis. The simplified reasoning can be summarized as follows: (i) genes that are co-mutated in a single cancer cell represent different pathways that cooperate during carcinogenesis; and, as a corollary, (ii) genes within the same pathway are never co-mutated.

9. Specific Co-Mutations

Table 2 lists the co-mutations of the polycistrons mir-17-20 and mir-106a-92. From this table, at least three observations can be made:

(1) both polycistrons mir-17-20 and mir-106a-92 have recurrent co-mutations;
(2) they share 10 co-mutations between them; and
(3) both polycistrons cooperate with co-mutations in at least three other (predicted) miRNAs.

If a genomic region is hit with retroviral insertions only few times in the entire screen, the chance of scoring an accidental co-mutation is lower. While a low frequency may also indicate low importance in tumorigenesis, it may simply reflect the mechanistic restrictions of retroviral insertion at that locus. If a region is hit frequently, the chance of false co-mutations increases. However, careful analysis of the region can minimize false co-mutation assignments. For example, if one only considers known or predicted genes, then in the present screen, there are 500 insertions near or into the Evi5 locus. Not only is this locus an area of preferred integration, the nearby Gfi locus also has similar high integration frequencies. On the one hand, polycistron mir-17-20 seemingly has 11 co-mutations in the Evi5 locus, and polycistron mir-106a-92 has 5. But a closer inspection of these integration sites reveal that the two polycistrons share (five and two, respectively) co-mutations in the 429 nt transcript AK037419, which represents an EST from the neonate thymus. Thus, transcript AK037419 cooperates with polycistrons mir-17-20 and mir-106a-92, respectively. This otherwise nondescript transcript itself is an oncogene as well. On the other hand, there clearly are integrations into the Evi5 gene as well: polycistron mir-17-20 has four co-mutations in intron 17 of Evi5, and polycistron mir-106a-92 has one in intron 16 of Evi5.

Another frequently hit region in the present screen is Notch1, with 248 integrations. In human T acute lymphatic leukemia, the mutations in the Notch1 locus are not evenly distributed but they fall into two broad groups which affect heterodimerization of the receptor and stability of the cytoplasmic signaling portion of the molecule³⁰. The mutations shown here fall into three broad groups, with 128 of the insertions into Notch1 in exon 34; these mutations presumably increase the stability of the cytoplasmic signaling portion³⁰. Two of these mutations are each co-mutated with mir-17-20 and mir-106a-92, respectively. As mentioned above more confidence can be placed into a co-mutation, if only a few integration sites are scored in the entire screen, and most or all of these integrations are in the tumors with the first mutation. Thus, another group has 12 mutations in intron 2 of Notch1. Two of these co-mutate with mir-17-20 upon closer inspection, the insertions into intron 2 are only 531 nt apart, and they coincide with transcript BF720900. This is an indication that mir-17-20 is co-mutated with transcript BF720900.

The set of regulatory RNAs and miRNAs that cause tumors when overexpressed, deleted or otherwise mutated are of particular interest in the present methods. The invention dramatically increases the number of known oncogenic regRNAs and miRNAs and is useful for the diagnosis and therapeutic treatment of human cancers.

### C. Diagnostic Methods and Reagents

In one aspect, the detection, identification, and quantitation of regRNA, including miRNA, and of mutations that affect the expression levels and/or function of these RNAs in tissue, body fluids, secretions and excretions are useful in cancer diagnostics are contemplate. Non-limiting examples include, but are not limited to (i) genotyping tumors for diagnosis, prognosis, and patient stratification in both therapy and clinical trials, and (ii) blood testing for early cancer detection of breast, ovary, colorectal and prostate cancer.

In one embodiment, an array (chip) containing complementary sequences of the regRNAs is used to score over- or under-expression of regRNAs in cancer tissue, which is linked to the cancer type and the precise diagnosis of it. This in turn allows better prognosis and therapy. In another embodiment of the invention, an oncogenic regRNA survey is carried out by the generally known methods of gel electrophoresis and detection by hybridization to complementary sequences. In yet another embodiment, DNA encoding regRNA is sequenced and mutations are recovered that may indicate non-physiological expression levels and/or function. When performed on bodily fluids, such as blood, these tests may be indicative of the presence of a tumor that escapes early detection by other means, or for which there are no early detection methods, or only detection methods that are more complicated and/or more expensive. Such tests may be carried out on material with or without prior amplification of nucleic acids.

### D. Therapeutic Methods

Those skilled in the art will, upon reading this disclosure, further understand how the disclosure of oncogenic regRNA including miRNA sequences and their co-mutations are useful in therapy. When over-expressed in cancer, the expression of such sequences may be repressed and the physiological state of the tumor cell may be restored, which, in turn prevents further proliferation. When under-expressed in cancer, the expression of such sequences may be supplemented and the physiological state of the tumor cell may be restored, which, in turn prevents further proliferation. When mutated in a way that changes the function, the mutated sequence may be corrected or eliminated. The delivery of drugs with these corrective effects may be accomplished by the known gene-therapy methods of transfection, infection and transduction.

In one general therapeutic method, molecules designed to bind specifically and with high affinity, e.g., by sequence-specific hybridization, may be may be employed to block overexpressed miRNA. For example, an oligonucleotide that targets a mature miRNA or its Drosha or Dicer cutting sites, may be employed for blocking levels or activity of a disease specific miRNA, as disclosed for example, in the Genetools website accessed at http://www.gene-tools.com/node/33.

### VI. Materials and Methods

### A. Generation of Tumors by Retroviral Mutagenesis in Mice with Chemically Mutagenized Paternal Haplotype

Cohorts of male BALB/c mice were injected three times with 0, 20, 50, 80 and 100 mg N-ethyl-N-nitrosourea (ENU)/kg body weight, with each injection one week apart³¹. The mice then became sterile, and the length of the sterility period was taken as a measure of the effectiveness of mutagenesis; only mice that had regained fertility after 11 weeks were used. After the sterility period the mice were mated with untreated BALB/cJ female mice to produce F1 pups. For each cohort infected with the SL3-3 virus³²⁻³⁵, the experiment involved four groups of mice, experimental group (E1) as well as three control groups (C1-C3). For E1, 2500 newborn (less than 36 hours old) pups were injected i.p. with retrovirus (both male and female pups were used). Control group C1: 200 newborn (less than 36 hours old) pups, male or female, from BALB/cJ (ENU-treated)×BALB/cJ crosses were mock-injected i. p., with medium alone. C2: 200 newborn (less than 36 hours old) pups, male or female, from non-treated BALB/cJ×BALB/cJ crosses were injected i. p. with retrovirus. C3: 100 newborn (less than 36 hours old) pups, male or female, from non-treated BALB/cJ×BALB/cJ crosses were mock-injected i. p., with medium alone. In all groups, mice were individually labeled 34 weeks after birth. Then the mice were weaned and tumors were allowed to develop. The average latency period was 85±31 days for SL3-3 virus, for tumors in mice with or without ENU mutagenesis of one parent. Once they became moribund due to cancer development, the mice were euthanized, gross necropsy was performed and tumor tissues were prepared.

### B. Viral Tap Recovery

To identify the integration sites of retroviral proviruses, the unknown flanking DNA was isolated using minor modifications of an anchored PCR method²⁰ Genomic tumor DNA from spleen or thymus was digested with enzyme 1, and a splinkerette adapter was ligated. This was followed by digestion of enzyme 2, to remove the internal viral fragment. The ligated DNA was amplified by PCR with adapter and virus-specific primers, followed by two additional PCR amplification steps with nested primers. The PCR product was purified by gel electrophoresis and sequenced. The sequence chromatograms were then fed into the bioinformatics pipeline for gene identification.

### C. Bioinformatics

The proviral inserts served as DNA tags for gene sequencing and identification. To extract and analyze genomic tags, a computational process was implemented. The input to this process was DNA sequencing chromatogram files, from which high-quality sequences were derived and matched to the mouse genome.

First, the sequence extraction step converted a chromatogram into a searchable tag sequence. The criteria for a searchable tag sequence include, but are not limited to, high-quality base-calls, non-vector sequence, non-repeat sequence and a length minimum. The base caller LifeTrace™ 36 was used to generate base calls from chromatograms and quality scores representing the accuracy of each base-call.

Second, using the base calls and quality scores from LifeTrace™, and a database of vector sequences, an algorithm was developed to automatically produce searchable sequences (i.e. sequences that can be matched to the mouse genome). In this algorithm, the region of high quality base calls was first determined by locating the longest stretch of base calls with a window-averaged quality score of 10. A window size of 11 was used to average the quality scores from five bases before to five bases after a central base-call. A quality score of ten indicated 90% accuracy of base calls.

Third, a database of vector sequences (entire retroviral genome sequences) was matched against the base calls to determine regions of viral sequence using the BLAST algorithm. Based on the sequencing construct, a stretch of less than 50 bases of viral sequence is expected on the 5′ end of the raw sequence; and read-through of short inserts can produce regions of 3′ viral sequence starting with a specific restriction site. If a region of high-quality, non-vector sequence longer than 32 bases remains, it becomes a searchable tag sequence.

Finally, a searchable tag sequence is a stretch of high-quality base calls that should be derived from the mouse genome. The MegaBLAST algorithm was used to search the mouse genome with each searchable sequence. A version of the mouse genome that has been “masked” for repeat sequences (both low-information local repeats and dispersed repetitive elements are not allowed for matches) was used at this step so that non-informative matches are not pursued. For each significant match to a tag sequence (there is usually only one, but occasionally there are more), 2 kb of unmasked genomic sequence is retrieved and realigned to the tag sequence. This realignment produces a more complete match in cases where the global search was interrupted by masked repetitive regions. Lastly, the latest annotation files from the (March 2005) UC Santa Cruz build of the mouse genome (mm6 or mm7) were used to locate nearby known and predicted genes. The genomic region into which the provirus inserted is displayed in the UC Santa Cruz Genome Browser (//genome.ucsc.edu).

### D. Algorithm to Identify miRNAs

To identify miRNAs, a method that takes advantage of the characteristic form of conservation profiles observed for most known miRNAs²⁶ was used. This form consists of a drop in conservation immediately flanking pre-miRNA regions. Mouse-human (mm6-hgl7) whole genome alignments from the UCSC Genome Bioinformatics website (genome.ucsc.edu) were used. For every position in the alignments, the percentage conservation in a 15 nucleotide window was calculated and assigned a value of 0 to 9 for 0% to 90% identity and “o” for 100% identity. The resulting conservation strings were then searched for a match to the following Perl™ regular expression:

/([0-8]{50,60})([o98]{53,260})([0-8]{50,60})/

Sequences that matched this were further analyzed with RNAfold³⁷⁻³⁹ to compute optimal secondary structures. The secondary structure output is in bracket notation where parentheses represent base pairings and periods are unpaired bases. A structure as an miRNA candidate was accepted if it matched the following Perl™ regular expression:

/((\((?:\.*\( ){24,})(\.{2,17}|\.*\({1,8}\.*\){1,8}\.*\({1,8}\.*){8}\.*)(\)(?:\.*\)){150,}))/x

This method detected 81% of all known mouse miRNAs.

### VII. EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Viral Tag Recovery and Locus Identification

Viral integrations sites (tags) were determined from tumors that were isolated and digested genomic tumor DNA, by using an anchored PCR technique as described above. This was performed by amplifying and sequencing a chimeric DNA fragment consisting of a short genomic sequence upstream of the viral 5′ LTR and part of the viral 5′ LTR itself. The tags were sequenced and mapped to the mouse genome sequence, and the affected transcription unit was determined. From 2373 tumors, 7300 tags were obtained, which mapped to 2,038 regions. Of these regions, 645 had two or more associated integration sites, with the largest region having 500 integrations.

### Example 2

### Expression Levels of regRNAs in Mouse Thymic Tumors

The RNA expression levels of three regRNAs were measured in mouse thymic tumors using quantitative methods with the results shown in FIGS. 5-7. Mouse tumors with integrations located in regions containing the regRNAs and control tumors (which lack such integrations) were examined by quantitative PCR using SYBR green. In all three regions, the majority of tumors have integrations which caused elevated expression of their respective noncoding RNAs.

The first region (R857:2) examined contains a group of noncoding transcripts located on chromosome 15, ˜50 kb downstream of the Myc gene (FIG. 5A). A primer set was designed to the 5′ end of AK030859 which is common to exon 1 of the other transcripts in the group. The sequence probed also falls within exon 1 of PVT1 (AK090048, plasmacytoma variant translocation 1), a region known for frequent chromosomal translocations⁴⁰. Twenty seven tumors with integrations in this area were assayed for AK030859 expression levels (see FIG. 5B for tumor locations). In 11 of 19 tumors containing integrations located within and downstream of AK030859, expression of AK030859 was elevated 5 to 40 fold over tumors with no integrations in this region (FIG. 5C).

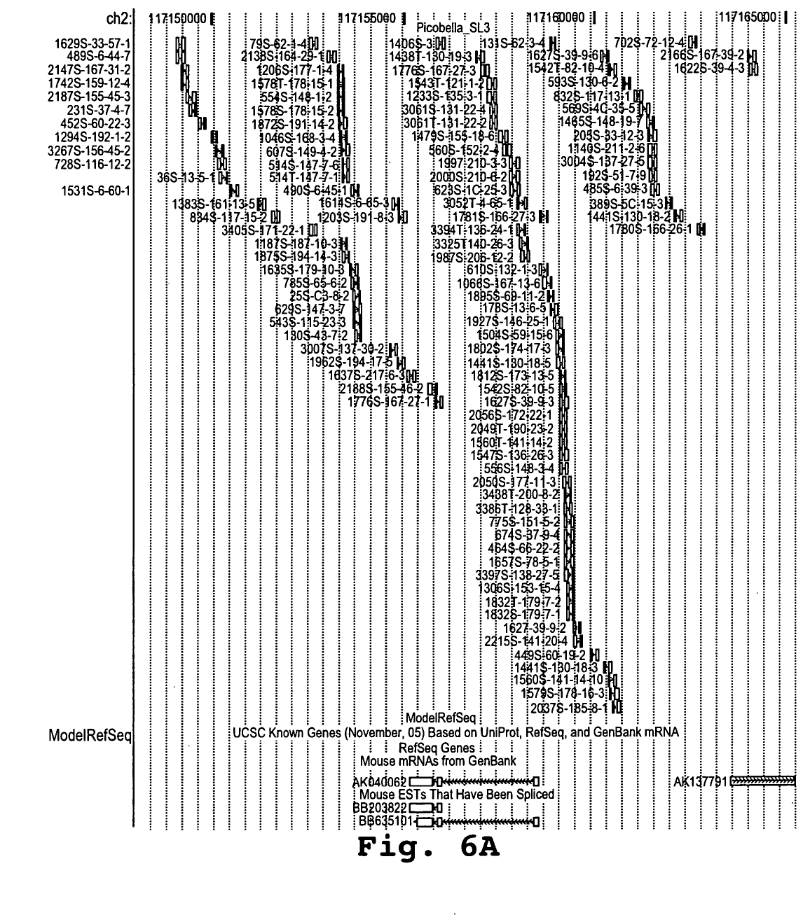

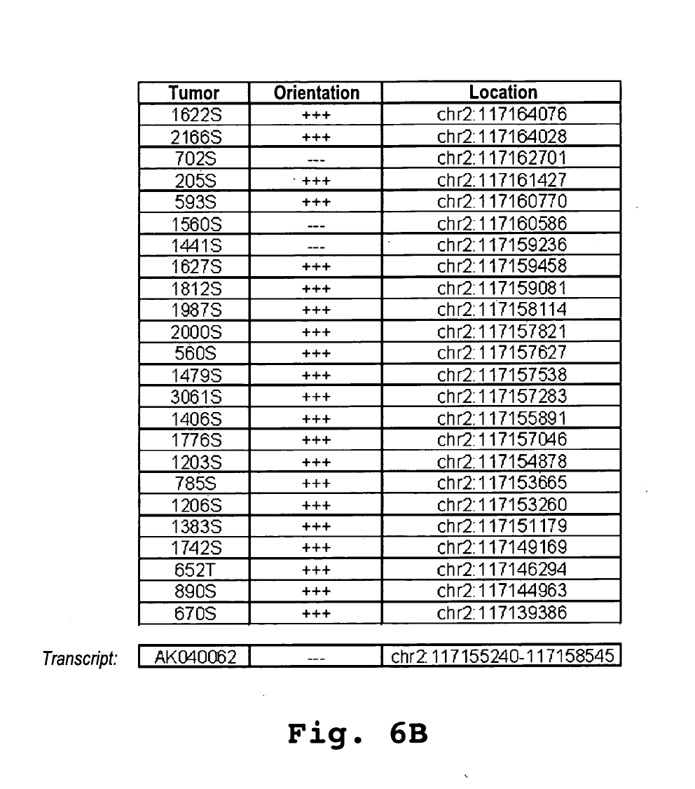

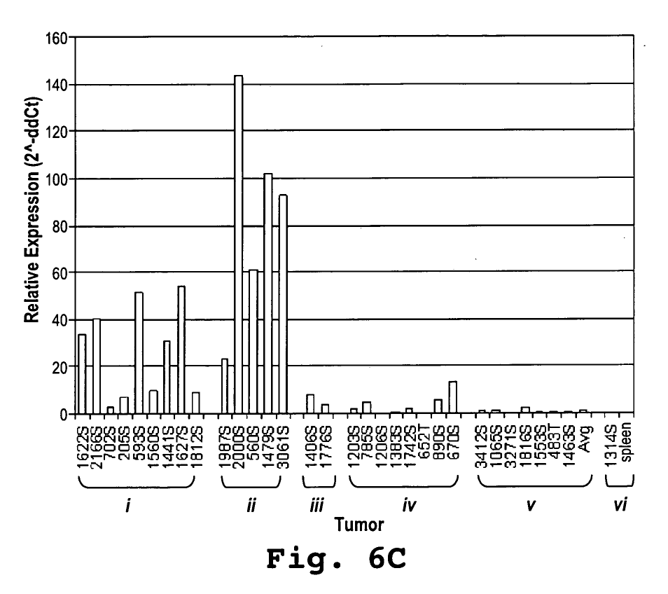

A second region (R894:1) with a high density of integration sites contains noncoding transcript AK040062 which is located on chromosome 2 (FIG. 6A). Primer sets were designed to AK040062 exon 2 and expression levels were measured for 24 tumors with integrations in this region (FIG. 6B). Elevated expression of AK040062 exon 2 was seen in tumors with integrations located upstream and within intron 1 of AK040062 (FIG. 6C). Of these 14 tumors, 10 had over 20 fold elevated expression of the noncoding RNA.

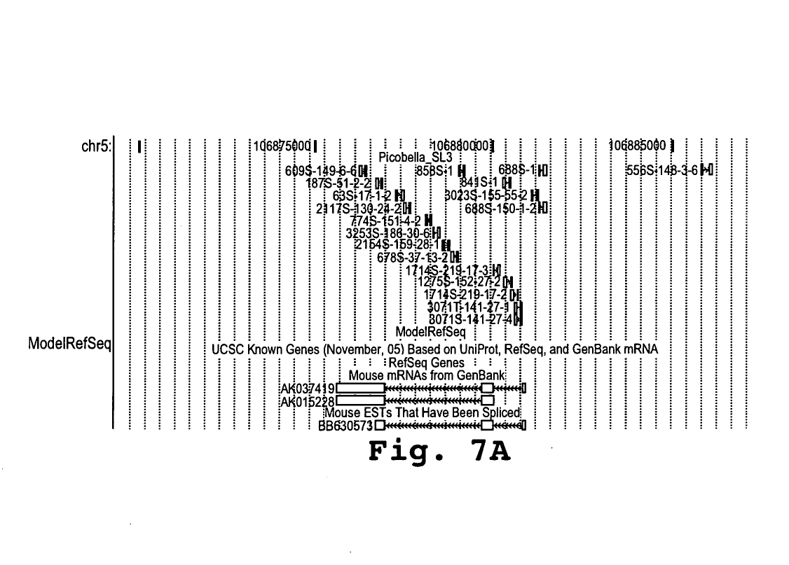

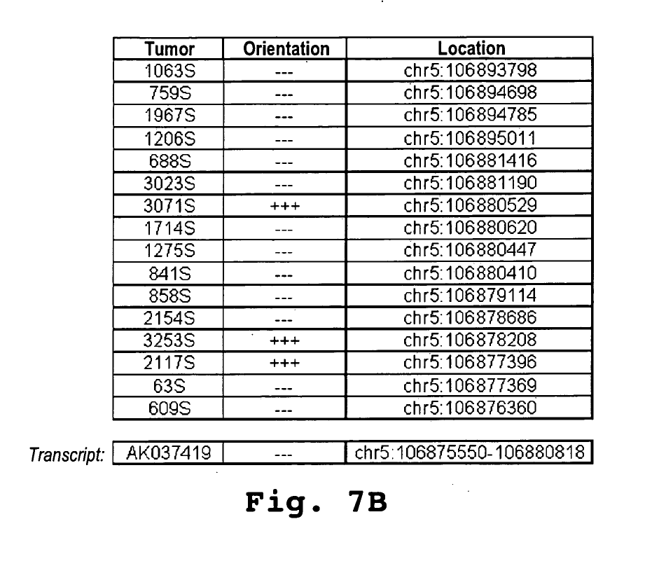

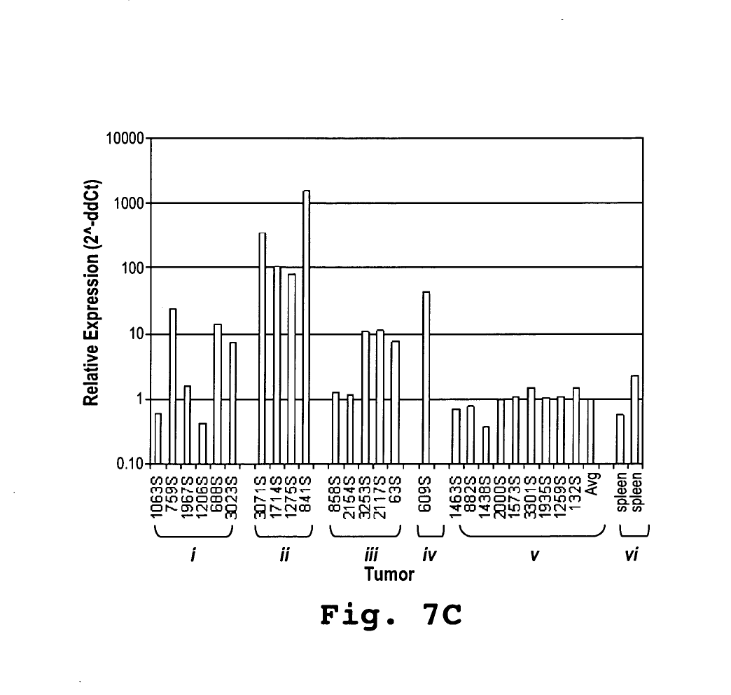

A third region (R217:3) examined for expression levels contains AK037419, a noncoding transcript located on chromosome 5, ˜15 kb downstream of the Gfi1 gene (FIG. 7A). Expression levels of AK037419 exon 3 were measured by qPCR in 16 tumors containing integration sites in this region (FIG. 7B). Expression of AK037419 exon 3 was increased between 7 to 1000 fold in 11 of the 16 tumors tested as compared to control tumors with no integrations in this region (FIG. 7C).

### Example 3

### Expression Levels of regRNAs in Human Tumors

The RNA expression levels of a newly identified regRNA (PVT1) was measured in human tumors using quantitative methods with the results shown in FIG. 8. The expression levels of PVT1 exon 1 were measured in matched human normal and cancer prostate RNA samples. Of nine matched tissue pairs, three tumor samples displayed 2 to 4 fold elevated expression of PVT1 exon1 as compared to their matched normal sample. Expression levels of PVT1 were measured by SYBR Green qPCR⁴¹ using primer sets designed to PVT1 exon 1.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

It is claimed:

1. A method for positively identifying a human miRNA sequence associated with a detectable disease state in humans, comprising
(i) identifying, from each of at least two animals having a detectable disease state produced by insertional mutation, the sequence of a genomic segment that is common to both animals, and that contains an insertional mutation,
(ii) identifying transcription units contained within the animal genome that are within about 200 Kbases, in either an upstream or downstream direction, of the sequenced genomic segment,
(iii) identifying human genomic transcription units that are orthologous to the transcription units identified in step (ii), and
(iv) for each human transcription unit identified in step (iii), employing a bioinformatics program capable of identifying putative miRNA sequences, to determine whether that transcription unit identified in step (iii) contains a putative miRNA sequence, in which case the putative miRNA sequence is positively identified as a human miRNA.

2. The method of claim 1, wherein the detectable disease state is a cancer, and step (i) is carried out by isolating the genomic segment from each of at least two animals having a detectable cancer.

3. The method of claim 1, wherein the detectable cancer is a lymphoma, and step (i) is carried out by isolating the genomic segment from each of at least two animals having a lymphoma.

4. The method of claim 1, wherein the insertional mutation in step (i) is a viral insertional mutation.

5. The method of claim 1, wherein the sequence identified in step (iii) is contained in a pri-miRNA.

6. The method of claim 1, wherein the sequence identified in step (iii) is contained completely within the mature miRNA.

7. An assay kit for diagnosing the presence or risk of cancer in a human subject comprising
a first reagent designed to react specifically with a human pri-miRNA or mature miRNA sequence identified in accordance with the method of claim 2, to form a first detectable reaction product, and
an indicator guide that indicates how the presence or amount of the reaction product correlates with the presence or risk of the disease state in a human subject.

8. The kit of claim 7, wherein the first reagent includes one of: (a) PCR reagents for detecting the presence or absence of the genomic sequence, and (b) oligonucleotide binding reagents for detecting the presence or absence of the genomic sequence.

9. The kit of claim 7, for use in diagnosing the presence of risk of a cancer in a human subject, wherein step (i) in the method of claim 1 is carried out by isolating the genomic fragment from each of at least two animals having a detectable cancer.

10. The kit of claim 9, for use in diagnosing the presence of risk of a lymphoma in a human subject, wherein step (i) in the method of claim 1 is carried out by isolating the genomic segment from each of at least two animals having a detectable cancer.

11. The kit of claim 1, wherein the first reagent is designed to react specifically with a mature human miRNA sequence identified in accordance with the method of claim 1.

12. A method of treating a cancer in a human subject comprising administering to the subject, a therapeutically effective amount of a compound capable of binding specifically to a mature human miRNA sequence identified in accordance with the method of claim 2.

13. An isolated mature human miRNA sequence selected from the group consisting of SEQ ID NOS: 1-55.

14. A method for identifying a human regulatory RNA (regRNA) sequence associated with a detectable disease state in humans, comprising
(i) identifying, from each of at least two animals having a detectable disease state produced by insertional mutation, the sequence of a genomic segment that is common to both animals, and that contains an insertional mutation,
(ii) identifying transcription units contained within the animal genome that are within about 200 Kbases, in either an upstream or downstream direction, of the sequenced genomic segment,
(iii) identifying human genomic transcription units that are orthologous to the transcription units identified in step (ii),
(iv) for each human transcription unit identified in step (iii), using a bioinformatics program to determine whether that transcription unit is a non-coding RNA sequence, and
(v) if the orthologous homologous human genomic sequence from step (iv) is a non-coding RNA sequence, classifying the sequence as a human regRNA sequence associated with the detectable disease state.

15. The method of claim 14, wherein the detectable disease state is a cancer, and step (i) is carried out by isolating the genomic segment from each of at least two animals having a detectable cancer.

16. The method of claim 14, wherein the human regRNA sequence is an miRNA, and step (iv) includes employing a bioinformatics program capable of identifying putative miRNA sequences to determine whether that transcription unit identified in step (iii) contains a putative miRNA sequence, in which case the putative miRNA sequence is positively identified as a human miRNA.

17. The method of claim 14, wherein the insertional mutation in step (i) is a viral insertional mutation.

18. The method of claim 14, which further includes utilizing the identified human regRNA sequence for diagnostic or therapeutic purposes.

19. An assay kit for diagnosing the presence or risk of cancer in a human subject comprising
a first reagent designed to react specifically with a human regulatory RNA (regRNA) sequence identified in accordance with the method of claim 15, to form a first detectable reaction product, and
an indicator guide that indicates how the presence or amount of the reaction product correlates with the presence or risk of the disease state in a human subject.

20. The kit of claim 19, wherein the first reagent includes one of: (a) PCR reagents for detecting the presence or absence of the genomic sequence, and (b) oligonucleotide binding reagents for detecting the presence of absence of the genomic sequence.
